(19) United States
(12) Patent Application Publication
(10) Pub. No.: US 2014/0294999 A1
(43) Pub. Date: Oct. 2, 2014

(54) NATURAL WEIGHT REDUCTION HEALTH COMPOSITION AND APPLICATION THEREOF

(71) Applicant: Huisen Li, (US)

(72) Inventors: Wei Sun, Guangzhou (CN); Haiyan Ou, Guangzhou (CN); Yiting Yang, Guangzhou (CN)

(73) Assignee: Infinitus (China) Company Ltd., Jiangmen, Guangdong (CN)

(21) Appl. No.: 14/129,548

(22) PCT Filed: Dec. 14, 2012
(86) PCT No.: PCT/CN2012/086652

§ 371 Date: Dec. 27, 2013

(30) Foreign Application Priority Data
Dec. 16, 2011 (CN) 201110424308.5

(51) Int. Cl. A61K 36/752; A61K 31/197; A61K 36/42

(52) U.S. Cl. CPC A61K 36/752; A61K 36/42; A61K 31/197
USPC 424/736

(57) ABSTRACT

Health-care composition made from the combination of herbal materials and a compound with a known effect of losing weight. The herbal materials are Balsam pear and dried tangerine peel and the known for losing weight is L-carnitine L-tartrate. Such combination not only achieves a comprehensive benefficiary effect on obesity but at the same time reduced the dosage of L-carnitine L-tartrate which is relative expensive, thus reducing the cost of product significantly.

### FIELD OF THE INVENTION

The present invention relates to the technical field of health-care food product, particularly to a composition for losing weight, which is prepared from Chinese herbal medicines and compound L-carnitine L-tartrate as the starting raw materials .

### BACKGROUND OF THE INVENTION

According to some investigation, in recent years, there are more than 90 million obese people and around 200 million overweight people in the current Chinese population. The market capacity of weight loss products is estimated to reach 60 billion Chinese Yuan in 2010. The proportion of obese and overweight population will increase as people's living style changes, and the market will continue to expand. Through years of clinical observation and research based on Traditional Chinese medicine(“TCM” hereinafter), the main reasons for obesity are found mainly as follows: 1) Obese patients have Spleen Deficiency (PiXu), often accompanied by the symptoms of Phlegm-Dampness and Qi Deficiency. Spleen is the foundation of acquired constitution and a source for generating and transporting Qi and Blood. Spleen Deficiency results in production of Dampness, accumulated Damp leads to production of Phlegm. When Qi is obstructed, the Phlegm is unable to be transported, leading to the tendency of Phlegm-Dampness, the tendency of Damp and Phlegm is common. According to the book entitled “Plain Questions.Treatise on Extraordinary diseases”, obese people have the tendency of Damp and Phlegm. Due to Spleen Deficiency and shortage of Qi, the Qi, Blood, Jing and body fluid are unable to be generated and transported, thus leading to obesity. 2) Most obese patients build up Heat inside, which is accumulated primarily in the Stomach. According to the book entitled “The theory of spleen and stomach”, when Heat is accumulated in the Spleen and Stomach, food is digested too quickly leading to constant hunger, and obesity is caused by a big appetite. Therefore, it makes obese patients difficult to control their appetites, and increases the difficulty and risk of controlling obesity. Thus, to prevent and alleviate the relevant symptoms of obese patients, it is necessary to begin with regulating and improving the function of Spleen and Stomach.

Meanwhile, among the available health-care food product for losing weight comprising L-carnitine

L-tartrate, the usage amount of L-carnitine L-tartrate is as high as 1.2-2 g per day, making the cost of the product quite high.

Traditional Chinese medicine emphases preventive treatment of disease and dietotherapy, thus, screening the raw materials having an effect of invigorating the Spleen and benefiting the Stomach from the pharmaceutical edible raw materials that have been used for thousands of years may provide a way of finding supplementary material to be used in combination with L-carnitine L-tartrate to achieve good weight loss effects while reducing the cost.

### SUMMARY OF THE INVENTIONS

The present invention aims at providing a natural health-care composition for losing weight, which comprises saponins, flavonoids and L-carnitine, being effective in regulating the functions of Spleen and Stomach according to TCM, inhibiting the synthesis of fat, accelerating the decomposition of fat and metabolism of carbohydrate, thus having a weight loss effect.

The present invention also aims at providing the use of aforesaid natural health-care composition for losing weight in the health-care food product having a weight loss effect.

The objects of the present invention are achieved by the following technical solutions: a natural health-care composition, being prepared from Balsam pear (Momordica charantia L., also known as “bitter gourd”), dried tangerine peel (Pericarpium Citri Reticulatae), and L-carnitine L-tartrate. Balsam pear and dried tangerine peel are herbal medicines commonly used in TCM.

In the present invention, the combination of Balsam pear and dried tangerine peel provides a composition containing large amounts of saponins and flavonoids compounds, and possesses the effects of invigorating the Spleen, dispelling Dampness and transforming Phlegm. In the combination, the Balsam pear has an effect of clearing Heat and dispelling Dampness, and contains an active ingredient (“Momordica charantia extract”) which has the effect of reducing the ingested fat and polysaccharide by about 40-60%, i.e., is able to inhibit the synthesis of fat and accelerate the metabolism of carbohydrate, and the dried tangerine peel has an effect of invigorating the Spleen and benefiting the Stomach and its effective ingredients are flavonoids capable of reducing the energy intake and energy utilization ratio, regulating the function of pancreatic β cells to regulate the metabolism of carbohydrate by secreting insulin, and improving the cholesterol catabolism in the body, thereby improving the body's obesity constitution. At the same time, L-carnitine L-tartrate can convert and make use of fat as an effective energy source, thus by burning excess fat accumulation to achieve the weight-loss effect. The present invention achieves the comprehensive weight loss effects of the aforesaid components that are complementary to each other.

Depending on the symptoms of obesity and the characteristics of preparation process, the present invention is generally prepared from the raw materials in the following weight portions: 2-50 portions of medicinal material Balsam pear, 1-20 portions of medicinal material dried tangerine peel, and 0.5-2 portions of L-carnitine L-tartrate, in which the medicinal materials Balsam pear and dried tangerine peel are extracted respectively, to obtain the extract of Balsam pear and the extract of dried tangerine peel, then L-carnitine L-tartrate is added and mixed evenly.

The present invention is preferably prepared from the raw materials in the following weight portions: 4-50 portions of medicinal material Balsam pear, 2-20 portions of medicinal material dried tangerine peel, and 0.5-1 portion of L-carnitine L-tartrate.

In the natural health-care composition for losing weight according to the present invention, the weight percentage of Balsam pear saponins is 1%-25%, the weight percentage of total flavonoids is 0.5%-5%, and the weight percentage of L-carnitine is 5%-60%.

Furthermore, the present invention provides the uses of aforesaid natural health-care composition for losing weight in the preparation of health-care food product having a weight loss effect. The weight loss composition prepared as above, when supplemented with pharmaceutically acceptable excipients and formulated into pharmaceutical dosage forms, would function as the health-care food having a weight loss effect, wherein the pharmaceutically acceptable excipients are, for example, magnesium stearate, microcrystalline cellulose, etc. Meanwhile, according to TCM dialectical method, said composition can be supplement with other Chinese herbal medicines regulating the Spleen and Stomach, dispelling Dampness and transforming Phlegm, so as to strengthen the effect of reducing weight through synergy.

The present invention provides the following advantages:

1. The raw materials used in the natural health-care composition for losing weight according to the present invention are originated from the common pharmaceutical edible raw materials and human nutritional supplements (thus lower toxicity), comprise a large amount of ingredients as saponins and flavonoids, can effectively regulate the functions of Spleen and Stomach of a person with obesity according to theories in TCM (improving overall health condition), and have the effects of invigorating the Spleen, dispelling Dampness and transforming Phlegm. In the time, they can inhibit the synthesis of fat, accelerate the decomposition of fat and metabolism of carbohydrate, thereby achieving a weight loss effect.

2. The health-care composition for losing weight according to the present invention preserves and enriches the functional ingredients having the effects of invigorating the Spleen and benefiting the Stomach, clearing Heat and dispelling Dampness sufficiently. It enables the effective ingredient in the raw material of Balsam pear to accelerate carbohydrate metabolism, by inhibiting the synthesis of fat. It enables the effective ingredient in the raw material of dried tangerine peel to improve the cholesterol catabolism in the body, by reducing the energy intake and energy utilization ratio, by regulating the function of pancreatic cells to regulate the metabolism of carbohydrate by secreting insulin. It also enables L-carnitine L-tartrate to assist in the cholesterol catabolism. Thus, the weight loss effect is achieved through the synergy of aforesaid pathways.

As the health-care composition for losing weight prepared according to the present invention achieves comprehensive inhibition on obesity and each active ingredient plays its respective role effectively, thus reducing the necessary amount of each material, in particular, the amount of L-carnitine L-tartrate. In other words, it reduces the cost of the product significantly.

The present invention will be further illustrated by the results of the following specific embodiments and pharmacodynamic studies.

### DETAILED DESCRIPTION OF PARTICULAR EMBODIMENTS OF THE INVENTION

The present invention is described by some specific embodiments illustrated as follows, herein it is necessary to point out that the following specific examples are only intended to further illustrate the present invention and shall not be deemed to limit the scope of the present invention. Some non-substantial modifications and adjustments made by the others according to the present invention still fall into the scope of the invention.

Part I: The composition of the natural health-care composition for losing weight

In the following examples, the medicinal materials Balsam pear and dried tangerine peel are extracted respectively by the following process, to obtain the extract of Balsam pear and the extract of dried tangerine peel:

Extract of Balsam pear: medicinal material Balsam pear is extracted by water or an organic solvent to obtain the Extract of Balsam pear. Wherein the organic solvent is an ethanol aqueous solution, the concentration of ethanol is in the range of 60-90%, the weight ratio of extraction solution to the medicinal material is in the range of 10:1-12:1, the temperature is as high as it keeps the solution boiling. Extraction is carried on 1-3 times, each duration lasts 1.5-2 h. Then the Extract is obtained through concentration and drying.

Extract of dried tangerine peel: medicinal material dried tangerine peel is extracted by water, alcohol or supercritical CO₂ to obtain the Extract of dried tangerine peel. The method is conventional, see Chinese Patents 201110023489.0 and 201110023459.X for details.

L-carnitine L-tartrate is a commercially available product, such as the L-carnitine L-tartrate manufactured by Lonza (China) Investments Co Ltd.

The health-care composition for losing weight of the present invention is composed by mixing the Extract of Balsam pear, the Extract of dried tangerine peel and L-carnitine L-tartrate, the weight percentage of Balsam pear saponins is 1%-25%, the weight percentage of total flavonoids is 0.5%-5%, and the weight percentage of L-carnitine is 5%-60%.

### Example 1

Raw materials: Balsam pear 4 g, dried tangerine peel 4 g, and L-carnitine L-tartrate 0.8 g.

Balsam pear is extracted by water or an organic solvent to obtain Extract of Balsam pear. Dried tangerine peel is extracted by water, alcohol or supercritical CO₂ to obtain Extract of dried tangerine peel. Then the Extract of Balsam pear, Extract of dried tangerine peel and L-carnitine L-tartrate are mixed evenly to obtain the health-care composition for losing weight.

### Example 2

Raw materials: Medicinal material Balsam pear 5 g, medicinal material dried tangerine peel 3 g, L-carnitine L-tartrate 0.8 g.

Balsam pear is extracted by water or an organic solvent to obtain Extract of Balsam pear. Dried tangerine peel is extracted by water, alcohol or supercritical CO₂ to obtain Extract of dried tangerine peel. Then the obtained Extract of Balsam pear, Extract of dried tangerine peel and L-carnitine L-tartrate are mixed evenly to obtain the health-care composition for losing weight.

### Example 3

Raw materials: Balsam pear 6 g, dried tangerine peel 2 gL-carnitine L-tartrate 1 g.

Balsam pear is extracted by water or an organic solvent to obtain Extract of Balsam pear. Dried tangerine peel is extracted by water, alcohol or supercritical CO₂ to obtain Extract of dried tangerine peel. Then the obtained Extract of Balsam pear, Extract of dried tangerine peel and L-carnitine L-tartrate are mixed evenly to obtain the health-care composition for losing weight.

### Example 4

Raw materials: Balsam pear 6 g, dried tangerine peel 4 gL-carnitine L-tartrate 1 g.

Balsam pear is extracted by water or an organic solvent to obtain Extract of Balsam pear. Dried tangerine peel is extracted by water, alcohol or supercritical CO₂ to obtain Extract of dried tangerine peel. Then the obtained Extract of Balsam pear, Extract of dried tangerine peel and L-carnitine L-tartrate are mixed evenly to obtain the health-care composition for losing weight.

### Example 5

Raw materials: Balsam pear 5 g, dried tangerine peel 5 gL-carnitine L-tartrate 1 g.

Balsam pear is extracted by water or an organic solvent to obtain Extract of Balsam pear. Dried tangerine peel is extracted by water, alcohol or supercritical CO₂ to obtain Extract of dried tangerine peel. Then the obtained Extract of Balsam pear, Extract of dried tangerine peel and L-carnitine L-tartrate are mixed evenly to obtain the health-care composition for losing weight.

### Example 6

Raw materials: Balsam pear 50 g, dried tangerine peel 20 gL-carnitine L-tartrate 2 g.

Balsam pear is extracted by water or an organic solvent to obtain Extract of Balsam pear. Dried tangerine peel is extracted by water, alcohol or supercritical CO₂ to obtain Extract of dried tangerine peel. Then the obtained Extract of Balsam pear, Extract of dried tangerine peel and L-carnitine L-tartrate are mixed evenly to obtain the health-care composition for losing weight.

### Example 7

Raw materials: Balsam pear 25 g, dried tangerine peel 10 gL-carnitine L-tartrate 1.5 g.

Balsam pear is extracted by water or an organic solvent to obtain Extract of Balsam pear. Dried tangerine peel is extracted by water, alcohol or supercritical CO₂ to obtain Extract of dried tangerine peel. Then the obtained Extract of Balsam pear, Extract of dried tangerine peel and L-carnitine L-tartrate are mixed evenly to obtain the health-care composition for losing weight.

### Example 8

Raw materials: Balsam pear 20 g, dried tangerine peel 10 gL-carnitine L-tartrate 0.5 g.

Balsam pear is extracted by water or an organic solvent to obtain Extract of Balsam pear. Dried tangerine peel is extracted by water, alcohol or supercritical CO₂ to obtain Extract of dried tangerine peel. Then the obtained Extract of Balsam pear, Extract of dried tangerine peel and L-carnitine L-tartrate are mixed evenly to obtain the health-care composition for losing weight.

The weight loss composition prepared in the aforesaid Examples 1-8, is further supplemented with pharmaceutically acceptable excipients and formulated into pharmaceutical dosage forms, wherein the pharmaceutically acceptable excipients are, for example, magnesium stearate, microcrystalline cellulose, etc.

Part II: The pharmacodynamic tests to demonstrate the weight loss effect of the health-care composition for losing weight

1. Experimental Site

College of Animal Science and Veterinary Medicine, Huazhong Agricultural University.

2. Experimental Purpose

Study the synergy effect of the natural health-care composition for losing weight according to the present invention, and confirm its weight loss effect.

3. Experimental Materials, Grouping, Dosage and Administration Method

3.1 Experimental Materials

(1) Aforesaid product as prepared in Example 3, referring to the amount of crude drug per kg human body weight, the administration amount is 8 g, once daily.

(2) Experimental animals: SPF SD rats, body weight being 180-200 g, male, provided by SHANGHAI SLAC LABORATORY ANIMAL CO. LTD. The quality certificate number of SD rat is SCXK (Shanghai) 2003-0003.

(3) Experimental Equipments and Reagents

I. Nissan Olympus automatic biochemical analyzer;

II. BIOTEK microplate reader;

III. Nissan KUBOTA KR-20000T low temperature high-speed centrifuge;

IV. EPPendorf 5415R centrifuge;

V. Sigma 1-14 centrifuge;

VI. Onetouch blood glucose meter and Onetouch blood glucose reagent, manufactured by U.S. Johnson & Johnson;

VII. HDL assay kit, manufactured by Biosino Bio-Technology and Science Incorporation.

3.2 Experimental grouping:

Three groups are assigned randomly with respect to body weight:

I. normal control group (also referred to as “normal group” or “negative control group”);

II. model control group (also referred to as “obesity model control group”, “model group” or “obesity model group”);

III. health-care composition for losing weight group (also referred to as “test group”, “test dosage group” or “complex factor for losing weight group”). Each group consists of 12 animals.

3.3 Experimental dosage:

Balsam pear and dried tangerine peel are calculated in the amount of crude material, the recommended dosage for human body is 8 g/60 kg/d, i.e., 133.4 mg/kg/d, the recommended dosage of L-carnitine L-tartrate for human body is 1 g/60 kg/d, i.e., 16.7 mg/kg/d. The equivalent dosage for rat is calculated as 10 times of the recommended dosage for human body, the dosage of Chinese herbal medicines is 1334 mg/kg/d, the dosage of L-carnitine is 167 mg/kg/d.

3.4 Administration Method

SPF SD male rats are randomly assigned into three groups: normal control group, obesity model control group, and health-care composition for losing weight group. The rats in normal control group are fed with basal diet, while the rats in obesity model control group and test group are fed with high-fat diet. The feeding time for establishing the models is 45 days. After the models have been successfully made, normal control group and obesity model group are fed with distilled water via gastric tube daily, while the test group is fed with the sample of test sample (i.e., the health-care composition) via gastric tube daily. All the data are processed for statistical analysis, and the effects of experimental treatment on the relevant indicators are observed. The duration of the trial is 45 days.

4. Data processing:

All the experimental data are represented as mean±SD, t test is performed between the data from two groups. All data are analyzed by statistical analysis software SPSS13.0. P<0.05 indicates a statistically significant difference.

5. Test Procedure and Results

5.1 The Effect of Experimental Treatment on Feed Intake

TABLE 1
The effect of experimental treatment on feed intake (g)

| | | Signifi- |
|---|---|---|
| Groups | Mean ± SD | cance |
| normal control group | 30.46 ± 1.67 | *** |
| obesity model group | 20.53 ± 1.73 | |
| health-care compositon for losing weight group | 22.04 ± 0.52 | NS |

Notes:
The normal group is compared with model group; the test group is compared with model group. “NS” indicates p > 0.05, “*” indicates P < 0.05, “**” indicates p < 0.01, “***” indicates p < 0.001, the same as follows.

From Table 1, it is seen that there is no significant difference in the feed intake between test group and model group, thus proves that the present invention does not affect the animal's feed intake.

5.2 The Effects of Experimental Treatment on Lee's Index and Body Weight

TABLE 2
The effect of experimental treatment on body weight (g)

| | | Signifi- |
|---|---|---|
| Groups | Mean ± SD | cance |
| normal control group | 393.40 ± 24.81 | *** |
| obesity model group | 466.02 ± 14.51 | |
| health-care compositon for losing weight group | 431.00 ± 14.82 | *** |

From Table 2, it is seen that the body weight of normal control group is lower than that of obesity model group very significantly. The body weight of the test group is lower than that of the model group very significantly, demonstrating the composition has a weight loss effect.

TABLE 3
The effect of experimental treatment on Lee's Index

| | | Signifi- |
|---|---|---|
| Groups | Mean ± SD | cance |
| normal control group | 24.43 ± 0.40 | *** |
| obesity model group | 25.80 ± 0.21 | |
| health-care composition for losing weight group | 25.43 ± 0.14 | ** |

From Table 3, it is seen that the Lee's Index of normal control group is lower than that of obesity model group very significantly. The experimental treatment in health-care composition for losing weight group can significantly improve the body condition of obesity in obese rats.

5.3 The Effect of Experimental Treatment on Visceral Fat Index

TABLE 4
The effect of experimental treatment
on the visceral fat index of rats

| | | Signifi- |
|---|---|---|
| Groups | Mean ± SD | cance |
| normal control group | 1.65 ± 0.40 | *** |
| obesity model groups | 3.66 ± 0.15 | |
| health-care composition for losing weight group | 2.84 ± 0.39 | *** |

From Table 4, it is seen that the visceral fat index of obesity model group is higher than that of normal control group very significantly. In comparison with obesity model group, the health-care composition for losing weight group can reduce the deposition of visceral fat very significantly.

5.4 The Effect of Experimental Treatment on Serum TG (Triglyceride)

TABLE 5
The effect of experimental treatment on serum TG (mmol/L)

| | | Signifi- |
|---|---|---|
| Groups | Mean ± SD | cance |
| normal control group | 0.52 ± 0.10 | *** |
| obesity model group | 1.13 ± 0.36 | |
| health-care composition for losing weight group | 0.58 ± 0.14 | *** |

From Table 5, it is seen that the serum TG of normal control group is lower than that of obesity model group very significantly, this result shows an injured lipid metabolism of rats in obesity model group, which demonstrates that the lipid metabolism of rats in obesity model group is abnormal. In the health-care composition for losing weight group, the injured lipid metabolism condition caused by obesity is improved very significantly.

5.5 The Effect of Experimental Treatment on Serum TC (Total Cholesterol)

TABLE 6
The effect of experimental treatment on serum TC (mmol/L)

| | | Signifi- |
|---|---|---|
| Groups | Mean ± SD | cance |
| normal control group | 1.20 ± 0.17 | *** |
| obesity model group | 1.63 ± 0.27 | |
| health-care compositon for losing weight group | 1.48 ± 0.18 | NS |

From Table 6, it is seen that the serum TC value of obesity model group is lower than that of normal control group significantly, thus shows an injured lipid metabolism of obesity model group. The serum TC of complex factor for losing weight group is not different from that of obesity model group, but the former shows a tendency of protection.

5.6 The Effect of Experimental Treatment on Rat Serum LDL

TABLE 7
The effect of experimental treatment on rat serum LDL (mmol/L)

| | | Signifi- |
|---|---|---|
| Groups | Mean ± SD | cance |
| normal control group | 0.59 ± 0.14 | *** |
| obesity model group | 0.89 ± 0.11 | |
| health-care composition for losing weight group | 0.71 ± 0.18 | NS |

From Table 7, it is seen that the serum LDL of obesity model group is lower than that of normal control group very significantly, the serum HDL level of complex factor for losing weight group is not different from that of obesity model group, but the former shows a tendency of protection.

5.7 The effects of experimental treatment on the rat serum cytokine levels of α-TNF, and IL-6

TABLE 8
The effect of experimental treatment on serum α-TNF, (ng/mL)

| Groups | Mean ± SD | P |
|---|---|---|
| normal control group | 59.84 ± 8.56 | *** |
| obesity model group | 120.21 ± 7.26 | |
| health-care composition for losing weight group | 85.29 ± 13.59 | * |

From Table 8, it is seen that the serum α-TNF, level of obesity model group is higher than that of negative control group very significantly, which shows that the experimental rats in the obesity model group are in an inflammatory status. The serum α-TNF, level of health-care composition for losing weight group is lower than that of obesity model group significantly, which shows that the health-care composition for losing weight can alleviate the inflammatory body status caused by obesity significantly.

TABLE 9
The effect of experimental treatment on serum IL-6 (pg/mL)

| Groups | Mean ± SD | P |
|---|---|---|
| normal control group | 19.42 ± 8.77 | *** |
| obesity model group | 94.18 ± 2.71 | |
| health-care composition for losing weight group | 72.56 ± 8.54 | ** |

From Table 9, it is seen that the serum IL-6 level of obesity model group is higher than that of negative control group very significantly, thus shows that the experimental rats in the obesity model group are in inflammatory status. The serum IL-6 level of health-care composition for losing weight group is lower than that of obesity model group very significantly, thus shows that the health-care composition for losing weight can alleviate the inflammatory body status caused by obesity significantly.

6. Experimental Conclusion

The aforesaid results of the instant experiment prove the weight loss effect of the natural health-care composition for losing weight according to the present invention. The weight loss effect is mainly achieved by improving the functional indicators such as serum TG, α-TNF, and IL-6, by regulating the disordered fat metabolism of obesity model, by alleviating the inflammatory status, hence reducing the accumulation of visceral fat. The combination of L-carnitine L-tartrate with two kinds of Chinese herbal medicines in the present invention not only achieves weight loss effect, but also reduces the amount of each raw material, in particular the amount of L-carnitine L-tartrate, thus reduces the cost of product significantly.

1-13. (canceled)

14. A composition for losing weight, comprising Balsam pear, dried tangerine peel and L-carnitine L-tartrate.

15. The composition according to claim 14, wherein Balsam pear is 2-50 portions by weight, dried tangerine peel is 1-20 portions by weight, and L-carnitine L-tartrate is 0.5-2 portion by weight.

16. The composition according to claim 14, wherein Balsam pear is 4-50 portions, dried tangerine peel is 2-20 portions, and L-carnitine L-tartrate 0.5-1 portion.

17. The composition according to claim 14, wherein Balsam pear is 4 portions, dried tangerine peel is 4 portions, and L-carnitine L-tartrate 0.8 portion.

18. The composition according to claim 14, wherein Balsam pear is 5 portions, dried tangerine peel is 3 portions, and L-carnitine L-tartrate 0.8 portion.

19. The composition according to claim 14, wherein Balsam pear is 6 portions, dried tangerine peel is 2 portions, and L-carnitine L-tartrate 1 portion.

20. The composition according to claim 14, wherein Balsam pear is 6 portions, dried tangerine peel is 4 portions, and L-carnitine L-tartrate 1 portion.

21. The composition according to claim 14, wherein Balsam pear is 5 portions, dried tangerine peel is 5 portions, and L-carnitine L-tartrate is 1 portion.

22. The composition according to claim 14, wherein Balsam pear is 50 portions, dried tangerine peel is 20 portions, and L-carnitine L-tartrate is 2 portion.

23. The composition according to claim 14, wherein Balsam pear is 25 portions, dried tangerine peel is 10 portions, and L-carnitine L-tartrate is 1.5 portion.

24. The composition according to claim 14, wherein Balsam pear is 20 portions, dried tangerine peel is 10 portions, and L-carnitine L-tartrate is 0.5 portion.

25. The composition according to claim 14, which comprises Balsam pear saponins 1%-25% by weight, total flavonoids 0.5%-5% by weight, and L-carnitine 5%-60% by weight.

26. The method of losing weight by administering an effective amount of the composition of claim 25.
